(19) United States
(12) Patent Application Publication
(10) Pub. No.: US 2024/0044901 A1
(43) Pub. Date: Feb. 8, 2024

(54) NEW METHOD TO PRONOSTIC LUNG CANCER

(71) Applicants: INSTITUT NATIONAL DE LA SANTÉ ET DE LA RECHERCHE MÉDICALE, Paris (FR); SORBONNE UNIVERSITÉ, Paris (FR); ASTRAZENECA AB, Södertälje (SE)

(72) Inventors: Marie-Caroline DIEU-NOSJEAN, Paris (FR); Claire GERMAIN, Paris (FR); Scott Alan HAMMOND, Gaithersburg, MD (US); Keith STEELE, Gaithersburg, MD (US)

(21) Appl. No.: 18/264,526

(22) PCT Filed: Feb. 8, 2022
(86) PCT No.: PCT/EP2022/052984

§ 371 Date: Aug. 7, 2023

(30) Foreign Application Priority Data
Feb. 9, 2021 (EP) 21305176.6

(51) Int. Cl. G01N 33/574

(52) U.S. Cl. CPC G01N 33/57423

(57) ABSTRACT

The present invention relates to the field of prognostic of lung cancer. In this study, the inventors used extensive gene expression profiling and flow cytometry for an integrative analysis of the phenotypes of the B cells and CD4+ T cells from tumors and blood of NSCLC patients by TLS-B density. They showed that TIL B cells and TIL CD4+ T cells are more highly activated in tumors than in the periphery and that they express all the ligand/receptor pairs necessary for B/T interactions and two-way co-stimulation. Moreover, a high density of TLS-B cells is associated with higher frequencies of activated CD4+ T cells and lower frequencies of both immune checkpoint (ICP)-expressing CD4+ T cells and regulatory CD4+ T cells (Tregs) in the intratumor CD4+ T cell compartment. High densities of TLS-B cells together with low densities of FoxP3+ CD3+ Tregs in NSCLC tumors consistently identified the group of patients with the best clinical outcome. Overall, these results suggest that TLS-B cells promote the development of protective CD4+ T cell-mediated immune responses. Thus, the present invention relates to a method for determining the TLS (Tertiary Lymphoid Structures) status of a patient suffering from a lung cancer and thus the survival time of said patient.

### FIELD OF THE INVENTION

The invention relates to a method for determining the TLS (Tertiary Lymphoid Structures) status of a patient suffering from a lung cancer and thus the survival time of said patient.

### BACKGROUND OF THE INVENTION

The tumor microenvironment is marked by its complexity and strong heterogeneity. Tumor-infiltrating lymphocytes (TILs) play a role in this heterogeneity and have an important impact on the clinical outcome of cancer patients. Studies have shown that while high densities of TIL CD8+ T cells or TIL B cells are associated with better survival in some cancers, other immune subsets, such as regulatory T cells (Tregs), are mostly associated with poor clinical outcomes.(1-3) Careful examination of the tumor microenvironment has pointed out the capacity of intratumor immune cells to organize into tertiary lymphoid structures (TLS),(4) specifically, ectopic lymphoid structures that appear upon sustained inflammation and are similar in organization to secondary lymphoid organs (SLOs). TLS have a B-cell area composed mainly of follicular CD20+ B cells (TLS-B cells) and CD21+ follicular dendritic cells (FDCs), adjacent to a T-cell area containing clusters of CD3+ T cells and mature DC-Lamp+ DCs (TLS-DC).(2,5,6) Antigen-specific responses develop in TLS, which are associated with increased severity in autoimmune diseases(7) but have a positive effect during infections by clearing pathogens.(8) In tumors, high TLS density is most often associated with better clinical outcomes.(4,9-11) We have previously showed that high densities of TLS-DC or TLS-B cells are associated with prolonged survival in non-small cell lung cancer (NSCLC);(2,5,12) and a TLS-B cell^{high} tumors are also linked to the development of tumor antigen-specific antibodies(2) and increased TIL CD4+ T cell repertoire clonality.(13)

### SUMMARY OF THE INVENTION

In this study, the inventors used extensive gene expression profiling and flow cytometry for an integrative analysis of the phenotypes of the B cells and CD4+ T cells from tumors and blood of NSCLC patients by TLS-B density. They showed that TIL B cells and TIL CD4+ T cells are more highly activated in tumors than in the periphery and that they express all the ligand/receptor pairs necessary for B/T interactions and two-way co-stimulation. Moreover, a high density of TLS-B cells is associated with higher frequencies of activated CD4+ T cells and lower frequencies of both immune checkpoint (ICP)-expressing CD4+ T cells and regulatory CD4+ T cells (Tregs) in the intratumor CD4+ T cell compartment. High densities of TLS-B cells together with low densities of FoxP3+ CD3+ Tregs in NSCLC tumors consistently identified the group of patients with the best clinical outcome. Overall, these results suggest that TLS-B cells promote the development of protective CD4+ T cell-mediated immune responses.

Thus, the invention relates to a method for determining the TLS (Tertiary Lymphoid Structures) status of a patient suffering from a lung cancer and thus the survival time of said patient. Particularly, the invention is defined by its claims

### DETAILED DESCRIPTION OF THE INVENTION

A first aspect of the invention relates to a method for determining the TLS (Tertiary Lymphoid Structures) status of a patient suffering from a lung cancer comprising i) determining in a sample obtained from said patient the expression level of at least one marker selected from the group consisting of CD69⁺, CM, CD8+, CD40L+, CD86+, TIM-3+, CD4+ and PD-1 ii) comparing the expression level determined at step i) with its predetermined reference value and iii) providing a good TLS status or a bad TLS status depending of the level of the expression level determined at step i) compared to its predetermined reference value.

According to the invention, the TLS are notably TLS-B or TLS-T cells.

According to the invention, the “expression level” of the markers of the invention also refers to the level or percentage of cells (B or T cells) which express at their surface these markers.

Thus, the invention also relates to a method for determining the TLS (Tertiary Lymphoid Structures) status of a patient suffering from a lung cancer comprising i) determining in a sample obtained from said patient the level of cells which express at least one marker selected from the group consisting of CD69⁺, CM, CD8+, CD40L+, CD86+, TIM-3+, CD4+ and PD-1 ii) comparing the level of cells positive for said at least one markers determined at step i) with its predetermined reference value and iii) providing a good TLS status or a bad TLS status depending of the level of the of cells expressing said markers determined at step i) compared to its predetermined reference value.

According to the invention, the cells expressing the markers of the methods of the invention are lived cells.

As demonstrated by the inventors (see the examples), determining the TLS status of a NSCLC patient is correlated with the phenotypic profile of B or T cells and thus will allow to identify the clinical outcome of said patient. A patient with a good TLS status, will have a good outcome and will not need a surgery and will better respond to the immune checkpoint inhibitors (9, 10, 11).

Thus, the invention also relates to a method for predicting the survival time of a patient suffering from a lung cancer comprising i) determining in a sample obtained from said patient the expression level of at least one marker selected from the group consisting of CD69⁺, CM, CD8+, CD40L+, CD86+, TIM-3+, CD4+ and PD-1 ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value.

In a particular embodiment, the invention also relates to a method for predicting the survival time of a patient suffering from a lung cancer comprising i) determining in a sample obtained from said patient the level of cells which express at least one marker selected from the group consisting of CD69⁺, CM, CD8+, CD40L+, CD86+, TIM-3+, CD4+ and PD-1 ii) comparing the level of cells positive for said at least one markers determined at step i) with its predetermined reference value and iii) providing a good prognosis status when the level determined at step i) is higher than its predetermined reference value, or providing a bad prognosis when the level determined at step i) is lower than its predetermined reference value.

Particularly, the expression level of these markers are determined in sample like peripheral-blood, serum, plasma or saliva.

In some embodiment, the expression level of a combination of different markers selected from the group consisting in: CD69+, CM, CD8+, CD40L+, CD86+, TIM-3+, CD4+ and PD-1 is determined.

Particularly, as the expression level of these markers can be done to determine the TLS status or the outcome of the patient suffering from a lung cancer, the following combination of the markers of the invention can be:

CM and CD8+; CD40L and CD8+; TIM-3+ and CD4+; TIM-3+ and CD8+; or TIM-3+, PD-1+ and CD8+.

Thus, when the expression levels of the markers or combination of markers CD69, or CM and CD8+, or CD40L+ and CD8+, or CD86+ will be higher than their predetermined reference value, a good TLS status will be provided.

According to the invention, the possible combinations are: CM and CD8+; or CD40L+ and CD8+.

In contrary, when the expression levels of the markers or combination of markers TIM-3+ and CD4+ or TIM-3+ and CD8+ or TIM-3+, PD-1+ and CD8+ will be lower than their predetermined reference value, a good TLS status will be provided. According to the invention, the possible combinations are: TIM-3+ and CD4+; or; TIM-3+ and CD8+; or TIM-3+, PD-1+ and CD8+.

Thus, the invention relates to a method for determining the TLS (Tertiary Lymphoid Structures) status of a patient suffering from a lung cancer comprising i) determining in a sample obtained from said patient the expression level of the markers or combination of markers CD69 or CM and CD8+ or CD40L+ and CD8+ or CD86+ii) comparing the expression level determined at step i) with its predetermined reference value and iii) providing a good TLS status when the expression levels determined at step i) are higher than its predetermined reference value, or providing a bad TLS status when the expression levels determined at step i) are lower than its predetermined reference value.

Thus, the invention relates to a method for determining the TLS (Tertiary Lymphoid Structures) status of a patient suffering from a lung cancer comprising i) determining in a sample obtained from said patient the expression level of the combination of markers TIM-3+ and CD4+ or TIM-3+ and CD8+ or TIM-3+, PD-1+ and CD8+ii) comparing the expression level determined at step i) with its predetermined reference value and iii) providing a good TLS status when the expression levels determined at step i) are lower than its predetermined reference value, or providing a bad TLS status when the expression levels determined at step i) are higher than its predetermined reference value.

According to the invention, the cells which express the marker CD69 are B cells and express also the marker CD19 and the marker CD20, the cells which express the markers CM and CD8+ are T cells and express also the marker CD3, the cells which express the markers CD40L+ and CD8+ are T cells and express also the marker CD3 and the cells which express the marker CD86+ are B cells and express also the marker CD19.

According to the invention, the cells which express the markers TIM-3+ and CD4+ are T cells and express also the marker CD3, the cells which express the markers TIM-3+ and CD8+ are T cells and express also the marker CD3 and the cells which express the markers TIM-3+, PD-1+ and CD8+ are T cells and express also the marker CD3.

The invention also relates to a method for predicting the survival time of a patient suffering from a lung cancer comprising i) determining in a sample obtained from said patient the expression level of the markers or combination of markers CD69 or CM and CD8+ or CD40L+ and CD8+ or CD86+ii) comparing the expression level determined at step i) with its predetermined reference value and iii) providing a good prognosis when the expression levels determined at step i) are higher than its predetermined reference value, or providing a bad prognosis when the expression levels determined at step i) are lower than its predetermined reference value.

The invention relates to a method for predicting the survival time of a patient suffering from a lung cancer comprising i) determining in a sample obtained from said patient the expression level of the combination of markers TIM-3+ and CD4+ or TIM-3+ and CD8+ or TIM-3+, PD-1+ and CD8+ii) comparing the expression level determined at step i) with its predetermined reference value and iii) providing a good prognosis when the expression levels determined at step i) are lower than its predetermined reference value, or providing a bad prognosis when the expression levels determined at step i) are higher than its predetermined reference value.

A second aspect of the invention relates to a method for determining the TLS (Tertiary Lymphoid Structures) status of a patient suffering from a lung cancer comprising i) determining in a sample obtained from the tumor of the patient the expression level of at least one marker selected from the group consisting of CD4+, TIGIT+, TIM-3+, HLA-DR+, CD25+, CD71+, PD-1+, 4-1BB+, GITR+, ICOS+, OX40+, CD38+ and CD69+ ii) comparing the expression level determined at step i) with its predetermined reference value and iii) providing a good TLS status or a bad TLS status depending of the level of the expression level determined at step i) compared to its predetermined reference value.

As demonstrated by the inventors (see the examples), determining the TLS status of a NSCLC patient is correlated with the phenotypic profile of B or T cells and thus will allow to identify the clinical outcome of said patient. A patient with a good TLS status, will have a good outcome and will not need a surgery will better respond to the immune checkpoint inhibitors (9, 10, 11).

Thus and according to the invention, the invention also relates to a method for predicting the survival time of a patient suffering from a lung cancer comprising i) determining in a sample obtained from the tumor of the patient the expression level of at least one marker selected from the group consisting of CD4+, TIGIT+, TIM-3+, HLA-DR+, CD25+, CD71+, PD-1+, 4-1BB+, GITR+, ICOS+, OX40+, CD38+ and CD69+ ii) comparing the expression level determined at step i) with its predetermined reference value and iii) providing a good prognosis or a bad prognosis depending of the level of the expression level determined at step i) compared to its predetermined reference value.

Thus the invention relates to a method for predicting the survival time of a patient suffering from a lung cancer comprising i) determining in a sample obtained from the tumor of the patient the expression level of at least one marker selected from the group consisting of CD4+, TIGIT+, TIM-3+, HLA-DR+, CD25+, CD71+, PD-1+, 4-1BB+, GITR+, ICOS+, OX40+, CD38+ and CD69+ ii) comparing the expression level determined at step i) with its predetermined reference value and iii) providing a good prognosis when the expression level determined at step i) is higher than its predetermined reference value, or providing a bad prognosis when the expression level determined at step i) is lower than its predetermined reference value.

Particularly and according to this second aspect of the invention, the expression levels of these markers are determined in a sample obtained from the tumor of the patient by biopsy.

According to the invention, the “expression levels of the markers of the invention” also refers to the level or percentage of cells (B or T cells) which express at their surface these markers.

Thus, the invention also relates to a method for determining the TLS (Tertiary Lymphoid Structures) status of a patient suffering from a lung cancer comprising i) determining in a sample obtained from the tumor of the patient the level of cells which express at least one marker selected from the group consisting of CD4+, TIGIT+, TIM-3+, HLA-DR+, CD25+, CD71+, PD-1+, 4-1BB+, GITR+, ICOS+, OX40+, CD38+ and CD69+ ii) comparing the level of cells positives for these markers determined at step i) with its predetermined reference value and iii) providing a good TLS status or a bad TLS status depending of the level of cells positives for these markers determined at step i) compared to its predetermined reference value.

Thus and according to the invention, the invention also relates to a method for predicting the survival time of a patient suffering from a lung cancer comprising i) determining in a sample obtained from the tumor of the patient the level of cells which express at least one marker selected from the group consisting of CD4+, TIGIT+, TIM-3+, HLA-DR+, CD25+, CD71+, PD-1+, 4-1BB+, GITR+, ICOS+, OX40+, CD38+ and CD69+ ii) comparing the level of cells positives for these markers determined at step i) with its predetermined reference value and iii) providing a good prognosis or a bad prognosis depending of the level of cells positives for these markers determined at step i) compared to its predetermined reference value.

In some embodiment, the expression level of a combination of different markers selected from the group consisting in: CD4+, TIGIT+, TIM-3+, HLA-DR+, CD25+, CD71+, PD-1+, 4-1BB+, GITR+, ICOS+, OX40+, CD38+ and CD69+ is determined.

Particularly, as the expression level of these markers can be done to determine the TLS status or the outcome of the patient suffering from a lung cancer, the following combination of the markers of the invention can be:

CD4+, TIGIT+ and TIM3+, or CD4+ and HLA-DR+, or CD4+ and CD25+, or CD4+ and CD71+, or CD4+ and TIM3+, or CD4+, PD-1+ and TIGIT+, or CD4+, 4-1BB+, GITR+, ICOS+ and OX40+, or CD4+ and GITR+, or CD4+, PD1+, TIGIT+ and TIM-3+, or CD4+, CD38+ and CD69+.

Thus, when the expression levels of the combination of markers CD4+, PD-1+ and TIGIT+; or CD4+, CD38+ and CD69+ will be higher than their predetermined reference value, a good TLS status will be provided.

In contrary, when the expression levels of the markers or combination of markers CD4+, TIGIT+ and TIM3+; or CD4+ and HLA-DR+; or CD4+ and CD25+; or CD4+ and CD71+; or CD4+ and TIM3+; or CD4+, 4-1BB+, GITR+, ICOS+ and OX40+; or CD4+ and GITR+; or CD4+, PD1+, TIGIT+ and TIM-3+ will be higher than their predetermined reference value, a poor TLS status will be provided.

Thus, the invention relates to a method for determining the TLS (Tertiary Lymphoid Structures) status of a patient suffering from a lung cancer comprising i) determining in a sample obtained from the tumor of said patient the expression level of the combination of markers CD4+, PD-1+ and TIGIT+, or CD4+, CD38+ and CD69+ ii) comparing the expression level determined at step i) with its predetermined reference value and iii) providing a good TLS status when the expression levels determined at step i) are higher than its predetermined reference value, or providing a bad TLS status when the expression levels determined at step i) are lower than its predetermined reference value.

Thus, the invention relates to a method for determining the TLS (Tertiary Lymphoid Structures) status of a patient suffering from a lung cancer comprising i) determining in a sample obtained from tumor of said patient the expression level of the combination of markers CD4+, TIGIT+ and TIM3+, or CD4+ and HLA-DR+, or CD4+ and CD25+, or CD4+ and CD71+, or CD4+ and TIM3+, or CD4+, 4-1BB+, GITR+, ICOS+ and OX40+, or CD4+ and GITR+, or CD4+, PD1+, TIGIT+ and TIM-3+ii) comparing the expression level determined at step i) with its predetermined reference value and iii) providing a good TLS status when the expression levels determined at step i) are lower than its predetermined reference value, or providing a bad TLS status when the expression levels determined at step i) are higher than its predetermined reference value.

Thus, the invention relates to a method for predicting the survival time of a patient suffering from a lung cancer comprising i) determining in a sample obtained from the tumor of said patient the expression level of the combination of markers CD4+, PD-1+ and TIGIT+, or CD4+, CD38+ and CD69+ ii) comparing the expression level determined at step i) with its predetermined reference value and iii) providing a good prognosis when the expression levels determined at step i) are higher than its predetermined reference value, or providing a bad prognosis when the expression levels determined at step i) are lower than its predetermined reference value.

Thus, the invention relates to a method for predicting the survival time of a patient suffering from a lung cancer comprising i) determining in a sample obtained from tumor of said patient the expression level of the combination of markers CD4+, TIGIT+ and TIM3+, or CD4+ and HLA-DR+, or CD4+ and CD25+, or CD4+ and CD71+, or CD4+ and TIM3+, or CD4+, 4-1BB+, GITR+, ICOS+ and OX40+, or CD4+ and GITR+, or CD4+, PD1+, TIGIT+ and TIM-3+ii) comparing the expression level determined at step i) with its predetermined reference value and iii) providing a good prognosis when the expression levels determined at step i) are lower than its predetermined reference value, or providing a bad prognosis when the expression levels determined at step i) are higher than its predetermined reference value.

According to the invention and the results obtained by the inventors, the marker FoxP3 can also be analysed to reinforce the methods of the invention. Thus, in a particularly embodiment, the FoxP3+CD3+ Tregs expression level is also determined for determining the TLS status of a patient suffering from a cancer or for predicting the survival time of a patient suffering from a cancer.

Thus, the invention relates to a method for determining the TLS (Tertiary Lymphoid Structures) status of a patient suffering from a lung cancer comprising i) determining in a sample obtained from the tumor of the patient the expression level of at least one marker selected from the group consisting of CD4+, TIGIT+, TIM-3+, HLA-DR+, CD25+, CD71+, PD-1+, 4-1BB+, GITR+, ICOS+, OX40+, CD38+CD69+ and FoxP3 ii) comparing the expression level determined at step i) with its predetermined reference value and iii) providing a good TLS status or a bad TLS status depending of the level of the expression level determined at step i) compared to its predetermined reference value.

According to the invention, the level of the marker FoxP3 is inversely correlated with the TLS status: when the TLS status is good the level of FoxP3 is low and when the TLS status is bad, the level of FoxP3 is high.

According to the invention, the lung cancer is a non-small-cell lung carcinoma (NSCLC).

As used herein, the term “patient” denotes a human with a lung cancer according to the invention.

As used herein; the term “CD69” denotes a human transmembrane C-Type lectin protein encoded by the CD69 gene. It is an early activation marker that is expressed in hematopoietic stem cells, T cells, and many other cell types in the immune system. It is also implicated in T cell differentiation as well as lymphocyte retention in lymphoid organs. Its Entrez reference number is: 969 and its UniProt reference number is: Q07108. According to the invention, the term “CD69+” denotes that the cell express at its surface the marker CD69. According to the invention, the cells expressing the CD69 marker are the B cells and T cells.

As used herein, the term “CM” for “Central Memory (cell)” denotes a subset of T lymphocytes among memory T cells. CM T cells are antigen-experienced T cells and readily proliferate and differentiate to effector memory T cells in response to antigenic stimulation.

They mainly home to T cell areas of lymphoid organizations such as secondary lymphoid organs or tertiary lymphoid structures (TLS), and to a lesser extend in peripheral blood. Their phenotype is CD45RA− CCR7+. Thus, according to the invention, the term “CM” denotes that this cell express at its surface the marker CCR7 but not CD45RA. According to the invention, the CM cells are the cells. In a particular embodiment, the phenotype of the CM cells is more precisely CD45RA− CD45RO+ CCR7+ CD62L+.

As used herein, the term “CD8” denotes a transmembrane glycoprotein that serves as a co-receptor for the T-cell receptor (TCR). Along with the TCR, the CD8 co-receptor plays a role in T cell signaling and aiding with cytotoxic T cell antigen interactions. Its Entrez reference number is: 925 and its UniProt reference number is: P01732. According to the invention, the term “CD8+” denotes that the cell express at its surface the marker CD8 and more particularly the marker CD8 alpha (which express the chain alpha of CD8). According to the invention, the cells expressing the CD8 marker are the T cells.

As used herein, the term “CD40L” also known as “CD154” denotes a protein that is primarily expressed on activated T cells and is a member of the TNF superfamily of molecules. It binds to CD40 (protein) express on antigen-presenting cells (APC), which leads to many effects depending on the target cell type. In total CD40L has three binding partners: CD40, α5β1 integrin and αIIbβ3. CD154 acts as a costimulatory molecule and is particularly important on a subset of T cells called T follicular helper cells (TFH cells). On TFH cells, CD154 promotes B cell maturation and function by engaging CD40 on the B cell surface and therefore facilitating cell-cell communication. A defect in this gene results in an inability to undergo immunoglobulin class switching and is associated with hyper IgM syndrome. Absence of CD154 also stops the formation of germinal centers and therefore prohibiting antibody affinity maturation, an important process in the adaptive immune system. Its Entrez reference number is: 959 and its UniProt reference number is: P29965. According to the invention, the term “CD40L+” denotes that the cell express at its surface the marker CD40L. According to the invention, the cells expressing the CD40L marker are the T cells.

As used herein, the term “CD86” denotes a protein expressed on dendritic cells, macrophages, B-cells, and other antigen-presenting cells. Along with CD80, CD86 provides costimulatory signals necessary for T-cell activation and survival. Depending on the ligand bound, CD86 can be used to signal for self-regulation and cell-cell association, or for attenuation of regulation and cell-cell disassociation. Its Entrez reference number is: 942 and its UniProt reference number is: P42081. According to the invention, the term “CD86+” denotes that the cell express at its surface the marker CD86. According to the invention, the cells expressing the CD86 marker are the B cells.

As used herein, the term “TIM-3” for “T-cell immunoglobulin and mucin-domain containing-3” or “HAVCR2” denotes a protein that in humans is encoded by the HAVCR2 gene. HAVCR2 was first described in 2002 as a cell surface molecule expressed on IFNγ producing CD4+Th1 and CD8+Tc1 cells. Later, the expression was detected in Th17 cells, regulatory T-cells, and innate immune cells (dendritic cells, NK cells, monocytes). Its Entrez reference number is: 84868 and its UniProt reference number is: Q8TDQ0. According to the invention, the term “TIM-3+” denotes that the cell express at its surface the marker TIM-3. According to the invention, the cells expressing the TIM-3 marker are the T cells.

As used herein, the term “CD4” denotes a glycoprotein found on the surface of immune cells such as T helper cells, monocytes, macrophages, and dendritic cells. It was discovered in the late 1970s and was originally known as leu-3 and T4 (after the OKT4 monoclonal antibody that reacted with it) before being named CD4 in 1984. In humans, the CD4 protein is encoded by the CD4 gene. Its Entrez reference number is: 920 and its UniProt reference number is: P01730. According to the invention, the term “CD4+” denotes that the cell express at its surface the marker CD4. According to the invention, the cells expressing the CD4 marker are the T cells.

As used herein, the term “PD-1” for “Programmed cell death 1” denotes a protein that in humans is encoded by the PDCD1 gene. PD-1 is express on the surface of cells that has a role in regulating the immune system's response to the cells of the human body by down-regulating the immune response and promoting self-tolerance by suppressing T cell inflammatory activity. This prevents autoimmune diseases, but it can also prevent the immune system from killing cancer cells. Its Entrez reference number is: 5133 and its UniProt reference number is: Q15116. According to the invention, the term “PD-1+” denotes that the cell express at its surface the marker PD-1. According to the invention, the cells expressing the PD-1 marker are the T cells.

As used herein, the term “TIGIT” for “T cell immunoreceptor with Ig and ITIM domains” denotes an immune receptor present on some T cells and Natural Killer Cells (NK).[It is also identified as WUCAM and Vstm3. TIGIT could bind to CD155 (PVR) on dendritic cells (DCs), macrophages, etc. with high affinity, and also to CD112 (PVRL2) with lower affinity. Research has shown that TIGIT-Fc fusion protein could interact with PVR on dendritic cells and increase its IL-10 secretion level/decrease its IL-12 secretion level under LPS stimulation, and also inhibit T cell activation in vivo. TIGIT's inhibition of NK cytotoxicity can be blocked by antibodies against its interaction with PVR and the activity is directed through its ITIM domain. Its Entrez reference number is: 201633 and its UniProt reference number is: Q495A1 According to the invention, the term “TIGIT+” denotes that the cell express at its surface the marker TIGIT. According to the invention, the cells expressing the TIGIT marker are the T cells.

As used herein, the term “HLA-DR” denotes an MHC class II cell surface receptor encoded by the human leukocyte antigen complex on chromosome 6 region 6p21.31. HLA-DR is a heterodimeric cell surface glycoprotein comprised of a 36 kDa alpha chain (heavy, also named HLA-DRA) and a 27 kD beta chain (light, also maned HLA-DRB), both anchored in the membrane. It is expressed on B cells, activated T cells, monocytes, macrophages, dendritic cells, and other cell types. In conjunction with the CD3/T cell receptor (TCR) complex and CD4 molecule, HLA-DR is critical for efficient peptide presentation to CD4+ T cells. The complex of HLA-DR (Human Leukocyte Antigen-DR isotype) and peptide, generally between 9 and 30 amino acids in length, constitutes a ligand for the T-cell receptor (TCR). HLA (human leukocyte antigens) were originally defined as cell surface antigens that mediate graft-versus-host disease. Identification of these antigens has led to greater success and longevity in organ transplant. According to the invention, the term “HLA-DR+” denotes that the cell express at its surface the marker HLA-DR. According to the invention, the cells expressing the HLA-DR marker are the T cells.

As used herein, the term “CD25” for “Interleukin-2 receptor alpha chain” denotes a protein that in humans is encoded by the IL2RA gene. The interleukin 2 (IL2) receptor alpha (IL2RA) and beta (IL2RB) chains, together with the common gamma chain (IL2RG), constitute the high-affinity IL2 receptor. Homodimeric alpha chains (IL2RA) result in low-affinity receptor, while homodimeric beta (IL2RB) chains produce a medium-affinity receptor. Normally an integral-membrane protein, soluble IL2RA has been isolated and determined to result from extracellular proteolysis. Alternately-spliced IL2RA mRNAs have been isolated, but the significance of each is currently unknown. Its Entrez reference number is: 3559 and its UniProt reference number is: P01589. According to the invention, the term “CD25+” denotes that the cell express at its surface the marker CD25. According to the invention, the cells expressing the CD25 marker are the T cells.

As used herein, the term “CD71” also known as “Transferrin receptor protein 1 (TfR1)” denotes an homodimeric transmembrane glycoprotein that in humans is encoded by the TFRC gene. TfR1 is required for iron import from transferrin into cells by endocytosis. CD71 plays a role in the control of cellular proliferation on many cell types including T and B cells. Its Entrez reference number is: 7037 and its UniProt reference number is: P02786. According to the invention, the term “CD71+” denotes that the cell express at its surface the marker CD71. According to the invention, the cells expressing the CD71 marker are the T cells.

As used herein, the term “4-1BB” also known as CD137 denotes a co-stimulatory immune checkpoint molecule. 4-1BB is expressed by activated T cells of both the CD4+ and CD8+ lineages. Although it is thought to function mainly in co-stimulating those cell types to support their activation by antigen presenting cells expressing its ligand (CD137L), CD137 is also expressed on dendritic cells, B cells, NK cells, neutrophils and macrophages. Its Entrez reference number is: 3604 and its UniProt reference number is: Q07011. According to the invention, the term “4-1BB+” denotes that the cell express at its surface the marker 4-1BB. According to the invention, the cells expressing the 4-1BB marker are the T cells.

As used herein, the term “GITR” for “glucocorticoid-induced TNFR-related protein” also known as “Tumor necrosis factor receptor superfamily member 18 (TNFRSF18)” denotes a protein that in humans is encoded by the TNFRSF18 gene. GITR is currently of interest to immunologists as a co-stimulatory immune checkpoint molecule. GITR is expressed on regulatory T cells and some activated immune cells including effector T cells, Natural Killer cells (NK) and neutrophils. GITR is critical for their development and activity of regulatory T cells. Its Entrez reference number is: 8784 and its UniProt reference number is: Q9Y5U5. According to the invention, the term “GITR+” denotes that the cell express at its surface the marker GITR. According to the invention, the cells expressing the GITR marker are the T cells.

As used herein, the term “ICOS” for “Inducible T-cell costimulatory” also known as CD278 denotes an immune checkpoint protein that in humans is encoded by the ICOS gene. CD278 or ICOS (Inducible T-cell COStimulator) is a CD28-superfamily costimulatory molecule that is expressed on activated T cells. It is thought to be important for Th2 cells in particular. ICOS is also essential for efficient interaction between T and B cells and for antibody responses to T-cell dependent antigens. Its Entrez reference number is: 29851 and its UniProt reference number is: Q9Y6W8. According to the invention, the term “ICOS+” denotes that the cell express at its surface the marker ICOS. According to the invention, the cells expressing the ICOS marker are the T cells.

As used herein, the term “OX40” also known as “Tumor necrosis factor receptor superfamily, member 4 (TNFRSF4)” and CD134 denotes a member of the TNFR-superfamily of receptors which is not constitutively expressed on resting naïve T cells, unlike CD28. OX40 is a secondary co-stimulatory immune checkpoint molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels. Its Entrez reference number is: 7293 and its UniProt reference number is: P43489. According to the invention, the term “OX40+” denotes that the cell express at its surface the marker OX40. According to the invention, the cells expressing the OX40 marker are the T cells.

As used herein, the term “CD38” also known as “cyclic ADP ribose hydrolase” denotes a glycoprotein found on the surface of many immune cells (white blood cells), including CD4+, CD8+, B lymphocytes and natural killer cells. CD38 also functions in cell adhesion, signal transduction and calcium signaling. Its Entrez reference number is: 952 and its UniProt reference number is: P28907. According to the invention, the term “CD38+” denotes that the cell express at its surface the marker CD38. According to the invention, the cells expressing the CD38 marker are the T cells.

As used herein, the term “survival time” denotes the percentage of people in a study or treatment group who are still alive for a certain period of time after they underwent a surgery to remove their tumor or a certain period of time after they were diagnosed with or started treatment for a disease, such as lung cancer (according to the invention). The survival time rate is often stated as a five-year survival rate, which is the percentage of people in a study or treatment group who are alive five years after their surgery or diagnosis or the start of treatment.

As used herein and according to the invention, the term “survival time” can regroup the term OS.

As used herein, the term “Overall survival (OS)” denotes the time from diagnosis of the cancer or the surgery (removal of the tumor) such as lung cancer (according to the invention) until death from any cause. The overall survival rate is often stated as a two-year survival rate, which is the percentage of people in a study or treatment group who are alive two years after their diagnosis or surgery or the start of treatment.

As used herein and according to the methods of the invention, the term “markers of the invention” denotes all the markers for which the expression levels is determined according to the above methods of the invention.

Measuring the expression level of the markers of the invention can be done by measuring the gene expression level of the markers of the invention or by measuring the protein level of the markers of the invention or by measuring the level (or percentage) of cells expressing the markers of the invention and can be performed by a variety of techniques well known in the art.

Typically, the expression level of a gene may be determined by determining the quantity of mRNA. Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Violet, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3 (4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6diclorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 nm (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6, 130, 101 and 6,716,979), Vio and VioBright (from Miltenyi), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649,138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can he produced that are identifiable based on their different spectral characteristics. For example, semiconductor nanocrystals can he produced that emit light of different colors based on their composition, size or size and composition. For example, quantum dots that emit light at different wavelengths based on size (565 nm, 655 nm, 705 nm, or 800 nm emission wavelengths), which are suitable as fluorescent labels in the probes disclosed herein are available from Life Technologies (Carlshad, Calif.).

Additional labels include, for example, radioisotopes (such as 3H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)), comparative genomic hybridization (CGH) or single cell RNASeq.

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat anti-avidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 nm) and a second specific binding agent (in this case an anti-DNP antibody, or antibody fragment, labelled with a second fluorophore (for example, a second spectrally distinct QUANTUM DOT®, e.g., that emits at 705 nm). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR (or q RT-PCR).

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Expression level of a gene may be expressed as absolute expression level or normalized expression level. Typically, expression levels are normalized by correcting the absolute expression level of a gene by comparing its expression to the expression of a gene that is not a relevant for determining the cancer stage of the patient, e.g., a housekeeping gene that is constitutively expressed. Suitable genes for normalization include housekeeping genes such as the actin gene ACTB, ribosomal 18S gene, GUSB, PGK1, TFRC, GAPDH, GUSB, TBP and ABL1. This normalization allows the comparison of the expression level in one sample, e.g., a patient sample, to another sample, or between samples from different sources.

According to the invention, the level of the proteins of the markers of the invention (“proteins of the invention”) may also be measured and can be performed by a variety of techniques well known in the art. For measuring the expression level of these proteins, techniques like Flow cytometry, CyTOF (mass cytometry), mass spectrometry, Western-blot or proteomic arrays (see below) allowing to measure the level of the membranous proteins are particularly suitable.

In the present application, the “level of protein” or the “protein level expression” or the “protein concentration” means the quantity or concentration of said protein. In another embodiment, the “level of protein” means the level of the protein fragments. In still another embodiment, the “level of protein” means the quantitative measurement of the proteins expression relative to a negative control.

Typically protein concentration may be measured for example by capillary electrophoresis-mass spectroscopy technique (CE-MS) or ELISA performed on the sample.

Such methods comprise contacting a sample with a binding partner capable of selectively interacting with proteins present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, capillary electrophoresis-mass spectroscopy technique (CE-MS), immunohistochemistry, immunohistofluorescent stainings, etc. The reactions generally include revealing labels such as fluorescent, chemioluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

In a particular embodiment, the detection of markers of the invention and more particularly the detection of the cells expressing the markers of the invention can be performed by flow cytometry (or CyTOF). When this method is used, the method consists of determining the amount of the markers of the inventions expressed on different cells. According to the invention and the flow cytometry method, when the fluorescence intensity is high or bright, the level of the markers express on the different cells is high and thus the expression level of the markers is high and when the fluorescence intensity is low or dull, the level of the markers express on the different cells is low and thus the expression level of the markers is low. According to the invention, the level of intensity of fluorescence will be determined thanks to an irrelevant antibody (=control antibody) which will allow to determine a threshold to determine if the fluorescence (signal) is positive or not.

Methods of the invention may comprise a step consisting of comparing the proteins and fragments concentration in circulating cells or the cells which express the markers of the invention with a control value. As used herein, “concentration of protein” refers to an amount or a concentration of a transcription product, for instance the proteins of the invention. Typically, a level of a protein can be expressed as nanograms per microgram of tissue or nanograms per milliliter of a culture medium, for example. Alternatively, relative units can be employed to describe a concentration. In a particular embodiment, “concentration of proteins” may refer to fragments of the proteins of the invention. Thus, in a particular embodiment, fragments of the proteins of the invention may also be measured. As used herein, the “level of cells which express the markers of the invention” or the “percentage of cells which express the markers of the invention” denotes an amount or quantity of cells which express, at their surface the markers of the invention. Thanks to different cohort and statistical analyse, the inventors determined a cut-of or threshold which allow to detect positive signal and thus which allow to determine a good or a bad TLS status depending of the level (or percentage) of positive cells expressing the markers of the invention.

As an example, predetermined reference values used for comparison of the expression levels may comprise “cut-off” or “threshold” values that may be determined as described herein. Each reference (“cut-off”) value for the levels of the markers of the invention may be predetermined by carrying out a method comprising the steps of:
a) providing a collection of samples from patients suffering of a lung cancer as described in the invention;
b) determining the level of the markers or the level of positive cells expressing the markers of the invention of the invention for each sample contained in the collection provided at step a);
c) ranking the tumor tissue samples according to said level
d) classifying said samples in pairs of subsets of increasing, respectively decreasing, number of members ranked according to their expression level,
e) providing, for each sample provided at step a), information relating to the actual clinical outcome for the corresponding cancer patient;
f) for each pair of subsets of samples, obtaining a Kaplan Meier percentage of survival curve;
g) for each pair of subsets of samples calculating the statistical significance (p value, Logrank test) between both subsets
h) selecting as reference value for the level, the value of level for which the p value is the smallest.

For example the expression level of the markers of the invention has been assessed for 100 cancer samples of 100 patients. The 100 samples are ranked according to their expression level. Sample 1 has the best expression level and sample 100 has the worst expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding lung cancer patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels.

Another example of method used to determine the optimal cut-off value is the Altman method (see reference 15).

In routine work, the reference value (cut-off value) may be used in the present method to discriminate lung cancer samples and therefore the corresponding patients.

Kaplan-Meier curves of percentage of survival as a function of time are commonly used to measure the fraction of patients living for a certain amount of time after the surgery (removal of the tumor) or treatment and are well known by the man skilled in the art.

The man skilled in the art also understands that the same technique of assessment of the expression level of a protein should of course be used for obtaining the reference value and thereafter for assessment of the expression level of a protein of a patient subjected to the method of the invention.

Such predetermined reference values of expression level may be determined for any protein defined above.

A further object of the invention relates to kits for performing the methods of the invention, wherein said kits comprise means for measuring the expression level of at least one of the markers of the invention or the percentage of cells expressing the markers of the invention in the sample obtained from the patient.

The kits may include nucleic probes, primers macroarrays, antibodies, microarrays or labelled ligand of the corresponding marker as above described. For example, the kit may comprise a set of probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards. Alternatively the kit of the invention may comprise amplification primers that may be pre-labelled or may contain an affinity purification or attachment moiety. The kit may further comprise amplification reagents and also other suitably packaged reagents and materials needed for the particular amplification protocol.

### Therapeutic Applications

The invention also relates to a method for treating a lung cancer in a patient with a bad prognosis as described above comprising the administration to said patient of an anti-cancer agent.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

Anti-cancer agent can be selected in the group consisting in cytarabine, anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agent may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies, fusion molecules (like antibodies fused to a drug or a cytokine) and small molecules like inhibitors of PKI.

Additional anti-cancer agent may be selected from, but are not limited to, growth or hematopoietic factors such as erythropoietin and thrombopoietin, and growth factor mimetics thereof.

In the present methods for treating cancer, additional therapeutic active agent can be added like an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiride, tetrahydrocannabinols, thiefhylperazine, thioproperazine and tropisetron. In a preferred embodiment, the antiemetic agent is granisetron or ondansetron.

In another embodiment, the further therapeutic active agent can be an hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In yet another embodiment, the further therapeutic active agent can be a checkpoint blockade cancer immunotherapy agent or a combination thereof.

Typically, the checkpoint blockade cancer immunotherapy agent is an agent which blocks an immunosuppressive receptor expressed by activated T lymphocytes, such as cytotoxic T lymphocyte-associated protein 4 (CTLA4), programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

Typically, the checkpoint blockade cancer immunotherapy agent is an antibody.

In some embodiments, the checkpoint blockade cancer immunotherapy agent is an antibody selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, anti-B7H6 antibodies, anti-4-1BB antibodies or anti-4-1BB ligand antibodies, anti-GITR antibodies, anti-ICOS antibodies or anti-ICOS ligand antibodies, anti-OX40 antibodies or anti-OX40 ligand antibodies or anti-CD40 antibodies or anti-CD40 ligand antibodies.

According to the invention, the anti-checkpoint blockade can be combined with another molecule like a cytokine or another anti-checkpoint blockade.

According to the invention, the immunotherapy agent can also be a CAR-T cells or a CAR-B cells which will express an anti-checkpoint blockade as described above.

Another aspect of the invention relates to a therapeutic composition comprising an anti-cancer treatment for use in the treatment of lung cancer in a patient with a bad prognosis as described above.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular, intrathecal or subcutaneous administration and the like or by inhalation.

Particularly, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

The invention will be further illustrated by the following figures and examples. However, these examples and figures should not be interpreted in any way as limiting the scope of the present invention.

### FIGURES

FIG. 1: Correlation between the percentage of PD-1+ Tim3+ CD8+ T cells or CM CD8+ T cells among total peripheral blood CD8+ CD3+ T cells and TLS-B cell density in the corresponding tumors. Correlations were evaluated by a Spearman test on a prospective cohort of 23 NSCLC patients. Abbreviations: CM, central memory; TLS-B, tertiary lymphoid structure-B cell.

FIG. 2: Correlations between TLS-B cell density and marker expression on TIL CD4+ T cells. Correlations between marker expression on TIL CD4+ T cells and TLS-B cell density. Markers significantly correlated with TLS-B cell density (FDR<0.05, 0.05<FDR<0.10, and up to 0.10<FDR<0.20) are shown. Vertical dashed lines represent limits for correlation factors (<−0.5 and >+0.5). Statistical test used: Spearman test.

FIG. 3: Correlation between TLS-B cell density and specific CD4+ T cell markers in tumors. Graphs represent the frequencies of cells expressing the mentioned markers among TIL CD3+CD4+ T cells with tumors stratified into TLS-B^{Low} (n=7, first quartile) and TLS-B^{High} (n=7, last quartile) groups. Means are indicated by horizontal red lines. P-values were calculated with the Mann Whitney test. * p<0.05. Abbreviations: ns, non-significant; TIL, tumor-infiltrating lymphocyte.

FIG. 4: Decreased frequencies of CD4+FoxP3+ Tregs in TLS-B^{high} tumors. Relative percentages of tumors with high (black) or low (white) frequencies of CD4+CD3+ Tregs in TLS-B^{low} and TL S-B^{high} groups. Abbreviations: NSCLC, non-small-cell lung cancer; TIL, tumor-infiltrating lymphocyte; TLS, tertiary lymphoid structure.

FIG. 5: High density of TLS-B cells cancels out the negative impact of high Treg density on overall survival. Kaplan-Meier curves of overall survival (OS) among the retrospective cohort (A, n=538 NSCLC patients; B-C, n=330 NSCLC patients), according to (A) CD20+ TLS-B cell density, (B) CD3+FoxP3+ Treg density, and (C) combined CD20+ TLS-B cell and CD3+FoxP3+ Treg densities (optimal cutoff value). The horizontal dotted line on each graph represents the median survival. Median survival values for each group of patients are also reported on the graph, as well as the difference in months between the best and worst surviving groups (Δ). P-values were determined with the log-rank test and corrected when indicated according to the formula proposed by Altman et al (15).

(A) thick line=TLS-B high; thin line=TLS-B low
(B) thick line=Tregs high; thin line=Tregs low
(C) thick line=TLS-B high Tregs low; thin line=TLS-B low Tregs high; thick dashed line=TLS-B high Tregs high; dotted thin line=TLS-B low Tregs low

TABLE 1
Correlation between specific peripheral blood T and B cell
subsets and density of TLS-B cells in NSCLC patients

| Subsets | Corr with TLS-B cells | P-value | FDR |
|---|---|---|---|
| CD69+ B | 0.53 | 0.06 | 0.52 |
| CM CD8+ T | 0.46 | 0.04 | 0.42 |
| CD40L+ CD8+ T | 0.43 | 0.07 | 0.53 |
| CD86+ B | 0.41 | 0.07 | 0.55 |
| TIM-3+ CD4+ T | −0.38 | 0.11 | 0.61 |
| TIM-3+ CD8+ T | −0.43 | 0.06 | 0.52 |
| TIM-3+ PD-1+ CD8+ T | −0.62 | 0.00 | 0.20 |

TABLE 2
Prognostic parameters for overall survival of NSCLC
patients in univariate analysis. The Cox regression
model was used for univariate analysis.

| | | Hazard | | |
|---|---|---|---|---|
| Variable | Class | ratio | 95% CI | P-value |
| Gender | Female | 1 | | |
| | Male | 1.268 | 0.958-1.677 | 0.097 |
| Age | Year | 2.239 | 1.406-3.565 | 0.00069 |
| | <60 | 1 | | |
| | ≥60 | 1.283 | 1.014-1.624 | 0.038 |
| Histological subtype | ADC | 1 | | |
| | SCC | 1.090 | 0.846-1.406 | 0.50 |
| | Others | 1.554 | 1.009-2.393 | 0.045 |
| pTNM stage | I | | | |
| | II | 1.349 | 1.012-1.797 | 0.041 |
| | III-IV | 2.841 | 2.190-3.685 | 3.77e−15 |
| Smoking history | Pack-Year | 1.027 | 0.960-1.1 | 0.44 |
| | <15 | 1 | | |
| | ≥15 | 1.193 | 0.835-1.707 | 0.33 |
| TLS-B cell density | Log | 0.711 | 0.599-0.845 | 0.00010 |
| | TLS-B^{low} | 1 | | |
| | TLS-B^{high} | 0.606 | 0.482-0.761 | 1.67e−05 |
| Treg density | Log | 1.071 | 0.983-1.166 | 0.1157 |
| | Treg^{low} | 1 | | |
| | Treg^{high} | 1.617 | 1.164-2.246 | 0.00420 |

A p-value <0.05 was considered statistically significant.
N = 538 patients for CD20+ TLS-B cell density,
n = 330 patients for CD3+ FoxP3+ Treg density.
Abbreviations: ADC, adenocarcinoma; SCC, squamous cell carcinoma; TLS, tertiary lymphoid structure.

TABLE 3
Multivariate Cox proportional hazards analysis for overall survival
of NSCLC patients. The Cox regression model was used for
multivariate analysis.

| | | Hazard | | |
|---|---|---|---|---|
| Variable | Class | ratio | 95% CI | P-value |
| Age | Year | 1.814 | 1.038-3.171 | 0.0366 |
| pTNM stage | I | | | |
| | II | 1.291 | 0.906-1.840 | 0.1578 |
| | III-IV | 2.620 | 1.899-3.614 | 4.47e−09 |
| TLS-B cell density | Log | 0.618 | 0.496-0.770 | 1.80e−05 |
| Treg density | Log | 1.119 | 1.025-1.222 | 0.0119 |

Parameters identified in univariate analysis as possibly influencing the clinical outcome (p < 0.05) were introduced into a multivariate Cox-proportional hazards regression model.
A p-value <0.05 was considered statistically significant.
N = 330 NSCLC patients.

### EXAMPLE

### Example 1: Determination of the TLS Status from Peripheral Blood

Material & Methods

Patients

This prospective study examined formalin-fixed, paraffin-embedded (FFPE) primary samples, fresh lung tumor samples and peripheral blood from 23 NSCLC patients who underwent surgery without any neoadjuvant chemoradiotherapy at Institut Mutualiste Montsouris and Cochin Hospital (Paris, France) to perform flow cytometry and immunohistochemistry analyses.

Immunohistochemistry

FFPE NSCLC blocks were selected, and sections were stained as previously described (2,12,14) under the antigen retrieval conditions and with the specific antibodies and reagents. Slides were digitally scanned with a Nanozoomer (Hamamatsu), operated with NDPview software.

Method for Cell Quantification

Cells from the entire tumor section were quantified with Calopix® software (Tribvn). Because TLS-B cells were highly aggregated (data not shown), their density was measured and expressed as a percentage of the whole tumor area:

[Total surface of CD20+TLS-B cells (mm²)/Total tumor area (mm²)]*100.

Both immunostaining and quantification were reviewed by at least two independent observers among.

Flow Cytometry

Mononuclear cells were recovered from fresh lung specimens or blood, and multiple-parameter flow cytometry analysis was performed as previously described (14) with the specific reagents and antibodies. Cells were acquired on an LSR Fortessa cell analyzer (BD Biosciences) by applying a gating strategy. Results were analyzed with DIVA (BD Biosciences) and FlowJo software (TreeStar, Inc) for Boolean analyses.

Statistical Analysis

Patients were stratified into two groups according to the density of TLS-B cell population high or low. This group was determined by using either the median density (0.623011805% for CD20+ TLS-B cells) or the optimal p-value approach (0.3256612% for CD20+ TLS-B cells), as previously described (2,12) (data not shown). Overall survival (OS) curves were estimated by the Kaplan-Meier method, and differences between patient groups were calculated with the log-rank test, corrected according to the formula proposed by Altman et al. (15). Univariate and multivariate analyses used the Cox regression model after testing the proportional hazards assumption (PHA P-value). Statistical analyses used StatView and R software. A p value <0.05 was defined as statistically significant.

In flow cytometry experiments, depending on data distribution (Shapiro normality test) and whether the observations were matched or not, the differences between the quantitative variables across the different groups were compared by the Kruskal-Wallis, one-way analysis of variance, Friedman, Mann-Whitney, or Wilcoxon (paired, non-Gaussian) tests, with appropriate adjustments for post-hoc comparisons (GraphPad Prism 5 software). A p-value <0.05 was defined as statistically significant. Correlations between immune subsets were evaluated by a Spearman test. A Benjamini-Hochberg correction was applied to determine the associated false discovery rates (FDRs).

Results:

A High Density of TLS-B Cells Correlates with Selective Peripheral Blood T and B Cell Subsets of NSCLC Patients

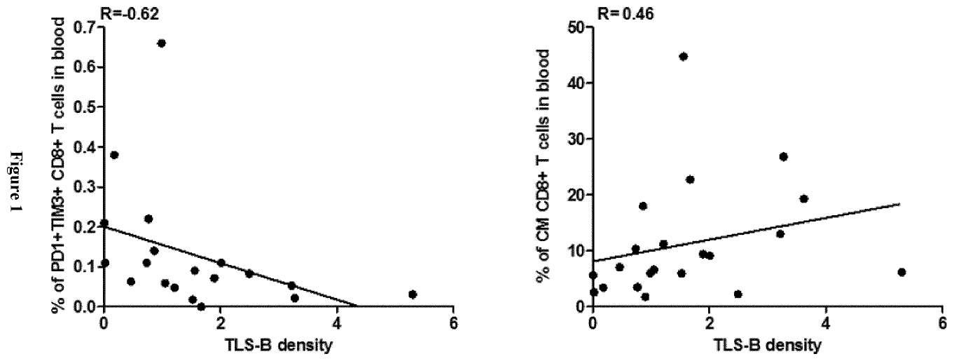

We examined the relation between the presence of TLS-B cells (assessed by immunohistochemistry in tumors) and the phenotype of fresh peripheral blood CD19+ B cells, CD3+CD4+ and CD3+CD8+ T cells (analyzed by flow cytometry) on the corresponding tumor biopsy. TLS-B cell density correlated positively with the percentages of activated CD69+ B cells and CD86+ B cells, as well as with central-memory (CD45RA− CCR7+) CD8+ T cells (FIG. 1, right panel; Table 1) and CD40L+ CD8+ T cells (Table 1). A contrario, TLS-B cell density correlated negatively with the percentages of TIM-3+ CD4+ T cells (Table 1), TIM-3+CD8+ T cells (Table 1), and TIM-3+ PD-1+ CD8+ T cells (FIG. 1, left panel, Table 1).

These results, taken together, emphasize that selective peripheral blood B and T cells are major indicators of tumor-associated TLS-B presence of NSCLC patients.

### Example 2: Determination of the TLS Status Based on the Quantification of Selective Tumor-Infiltrating CD4+ T Cell Subsets of NSCLC Patients

Material & Methods

Patients

This retrospective study examined formalin-fixed, paraffin-embedded (FFPE) primary NSCLC samples from 538 patients who underwent surgery without any neoadjuvant chemoradiotherapy between 2001 and 2005 at Hôtel Dieu Hospital in Paris, France (data not shown). We also used 56 fresh tumor biopsies, retrieved prospectively at the Institut Mutualiste Montsouris and Cochin Hospital (Paris, France), to study gene expression (n=26) or perform flow cytometry analyses (n=30, associated when possible with non-tumor lung biopsies and/or peripheral blood analyses) (data not shown). Patients who had received neoadjuvant chemoradiotherapy were ineligible. Serial sections of the corresponding FFPE NSCLC tumors were obtained for these 61 patients, together with a written informed consent before their inclusion in the study. The local ethics and human investigation committees (no 2008-133, 2012-0612, and 2017-A03081-52) approved these protocols, in application of article L.1121-1 of the French Public Health Code. Peripheral blood was obtained from healthy volunteers at the Etablissement Frangais du Sang (EFS, Paris, France, n° 15EFS012 and n° 18EFS033).

Immunohistochemistry

FFPE NSCLC blocks were selected, and sections were stained as previously described (2,12,14) under the antigen retrieval conditions and with specific antibodies and reagents. Slides were digitally scanned with a Nanozoomer (Hamamatsu), operated with NDPview software.

Method for Cell Quantification

Cells from the entire tumor section were quantified with Calopix® software (Tribvn). Because TLS-B cells were highly aggregated (data not shown), their density was measured and expressed as a percentage of the whole tumor area:

[Total surface of CD20+TLS-B cells (mm²)/Total tumor area (mm²)]*100.

CD3+FoxP3+ T cells (data not shown) were described by their cell density, i.e., the absolute number of cells/mm2 of tumor area, as previously reported.(12) Both immunostaining and quantification were reviewed by at least two independent observers.

Flow Cytometry

Mononuclear cells were recovered from fresh lung specimens or blood, and multiple-parameter flow cytometry analysis was performed as previously described,(14) with the specific reagents and antibodies. Cells were acquired on an LSR Fortessa cell analyzer (BD Biosciences). Results were analyzed with DIVA (BD Biosciences) and FlowJo software (TreeStar, Inc) for Boolean analyses. Multiple phenotypes were represented as pie charts, with Pestle and Spice software (Mario Roederer, NIAID).

Cell Sorting and Gene Expression Analysis

CD3+CD4+CD8− T cells were sorted from lung tumor mononuclear cells on a FACS Aria III cell sorter (BD Biosciences) as previously described,(13,14) with specific reagents and antibodies. Purity was >98%. Total RNA was extracted with the RNeasy Mini Kit (Qiagen SAS, Courtaboeuf, France). Digital multiplexed gene expression analysis used the NanoString nCounter system (PanCancer Immune Profiling Panel, NanoString Technologies), with 4 ng of total RNA from each sample, after pre-amplification, as previously described.(13,14) Genes with geomean counts before normalization below a threshold determined on background, i.e., less than 20 geomean counts, were excluded from subsequent analysis. Raw data were normalized with nSolver software (NanoString Technologies), based on the 10 most relevant of 39 housekeeping genes.

Statistical Analysis

For each cell population of interest, patients were stratified into two groups according to its density—high or low. This group was determined by using either the median density (0.623011805% for CD20+ TLS-B cells and 39.211937000 cells/mm² for CD3+FoxP3+ Tregs) or the optimal p-value approach (0.3256612% for CD20+ TLS-B cells, and 21.93277 cells/mm² for CD3+FoxP3+ Tregs), as previously described (2,12) (data not shown). Overall survival (OS) curves were estimated by the Kaplan-Meier method, and differences between patient groups were calculated with the log-rank test, corrected according to the formula proposed by Altman et al.(15) Univariate and multivariate analyses used the Cox regression model after testing the proportional hazards assumption (PHA P-value). Statistical analyses used StatView and R software. A p value <0.05 was defined as statistically significant.

In flow cytometry experiments, depending on data distribution (Shapiro normality test) and whether the observations were matched or not, the differences between the quantitative variables across the different groups were compared by the Kruskal-Wallis, one-way analysis of variance, Friedman, Mann-Whitney, or Wilcoxon (paired, non-Gaussian) tests, with appropriate adjustments for post-hoc comparisons (GraphPad Prism 5 software). A p-value <0.05 was defined as statistically significant.

In the Nanostring analysis, correlations were evaluated by a Spearman test. A Benjamini-Hochberg correction was applied to determine the associated false discovery rates (FDRs). Only FDRs<0.10 were considered statistically significant.

Results:

A High Density of TLS-B Cells is Associated with a Specific Intratumor CD4+ T Cell Gene Expression Signature

As our previous study showed that the presence of TLS-B cells in the tumor microenvironment mainly favors intratumor CD4+ T cell clonal expansion,(13) we analyzed the expression of 550 immune-related genes in sorted TIL CD4+ T cells from 26 NSCLC patients in relation to their TLS-B cell densities.

TLS-B cell density was correlated with 11 genes expressed by TIL CD4+ T cells (data not shown). Among them, 3 genes were positively correlated with TLS-B cell density: LY96 (Lymphocyte antigen 96), despite its weak expression (data not shown); MERTK, a tyrosine kinase receptor involved in T cell survival and differentiation following TCR activation; and POU2AF1 (POU class 2 associating factor 1), a transcriptional co-activator also induced after TCR triggering and contributing to germinal center (GC) formation, to IFN-γ and IL-2 promoter activities,(16) and required for robust CD4+ T cell memory responses. Conversely, eight genes were negatively correlated with TLS-B cell density (data not shown). Four of them were highly expressed by CD4+ T cells (data not shown): PIK3CG and MAP2K1, both involved in cell growth, survival, and proliferation; ITGB1, related to cell adhesion; and CD5, which plays a role in Treg differentiation.(17) The other four genes encoded the adhesion molecule CD58; the chemokine XCL2; the transcription factor POU2F2; and DPP4, a receptor involved in TCR-mediated T cell co-activation and Treg-mediated immunosuppression.

Taken together, these results demonstrate that TLS-B cell density correlates with a specific transcriptional gene signature associated with Th1/memory response and GC formation, as well as with decreased proliferative capacities, chemotaxis, and Treg functions in tumor-infiltrating CD4+ T cells.

Specific Phenotypic Profile of Tumor-Infiltrating B Cells Compared with B Cells at Non-Tumor Sites

We then analyzed the expression of cell surface molecules by TIL-B cells in lung tumors compared with those at distant sites, i.e., non-tumor (NT) lung sections and blood samples from NSCLC patients and blood from healthy individuals. Most B cells expressed MHC-class II (data not shown) and CD40 (data not shown) molecules in all the tissues we studied. Consistent with the greater frequency of memory B cells and plasma cells in tumors than in blood (data not shown), a higher percentage of CD27+ B cells was also detected in tumors than in blood (data not shown). The percentages of B cells expressing CD69 (data not shown) (mostly activated IgD-memory B cells, data not shown panels), and CD86 (data not shown) (mainly transitional B cells and GC B cells, data not shown) were significantly higher in tumors than blood. Of note, the expression of CD80 and CD83 remained similar at the different sites (range=10-20%). By contrast, the percentage of ICOS-L+B cells dropped dramatically in tumors compared with NT sites (data not shown) and was negatively associated with TLS-B cell density (data not shown). Consistently and as in NSCLC lymph nodes, very low percentages of ICOS-L+ naive and transitional pre-GC IgD+B cells were observed in tumors than in NSCLC patient blood samples (data not shown). Finally, regulatory B cells, defined as CD38^{high}CD24^{high}, were barely detected in tumors (<0.1% of total B cells), compared with NT sites (data not shown).

Expression of Activation, Co-Stimulatory Receptors, and ICPs by TIL CD4+ T Cells

We then analyzed and compared the expression of activation, co-stimulatory and immunosuppressive molecules by CD4+ T cells in NSCLC versus NT sites.

As previously observed,(12) there was a much greater frequency of effector-memory (EM) CD4+ T cells was observed in tumors than in NSCLC NT sites or in blood from healthy donors (HD) (data not shown). The percentage of CD4+ T cells not expressing activation markers was very low in tumors compared with NT lung and NSCLC blood (data not shown). Higher percentages of cells expressing HLA-DR, CD69, CD38, and CD71 were detected in tumors than in NT lung and blood from healthy donors and NSCLC patients (data not shown). Of intratumor CD4+ T cells, 78% expressed CD69, and half were triple positive CD69+CD38+HLA-DR+. Although the percentage of CD25+ cells was similar between the different sites, most CD25+ T cell subsets in tumors, contrary to those in blood, co-expressed several activation markers (data not shown).

The percentage of CD4+ T cells expressing no co-stimulatory receptors was significantly lower in tumors than in either NT lung or NSCLC blood (44% versus 68% and 74%, respectively; data not shown). In particular, CD4+ T cells were positive for ICOS, OX40, 4-1BB, and GITR more frequently in tumors than in the other two sites. Interestingly, only 35% of CD4+ T cells in tumors did not express ICP, compared with those from NT lung (almost 65%) and blood (more than 75%) (data not shown). The percentages of cells expressing at least one ICP were higher in tumors than at NT sites (except for BTLA).

Taken together, these results demonstrate that CD4+ T cells expressing molecules involved in B-T cell interactions (i.e., co-activation and inhibitory receptors) are present in higher percentages in tumors than at NT sites.

A High Density of TLS-B Cells is Associated with Lower Frequencies of Immune Checkpoint-Expressing CD4+ T Cells in Tumors

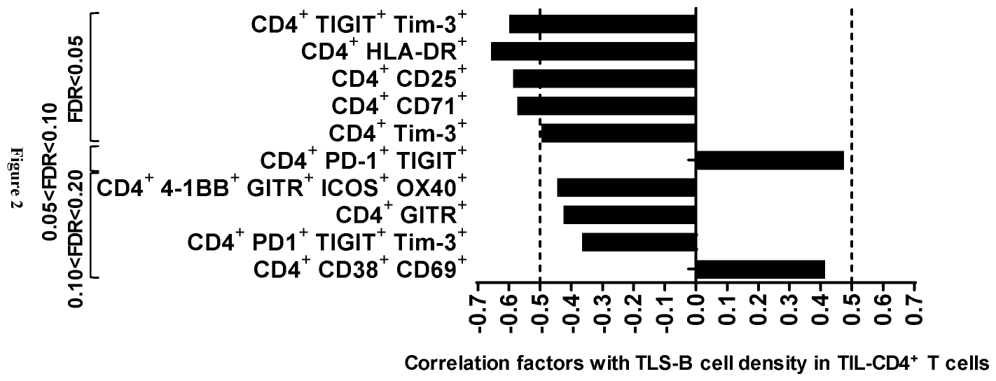

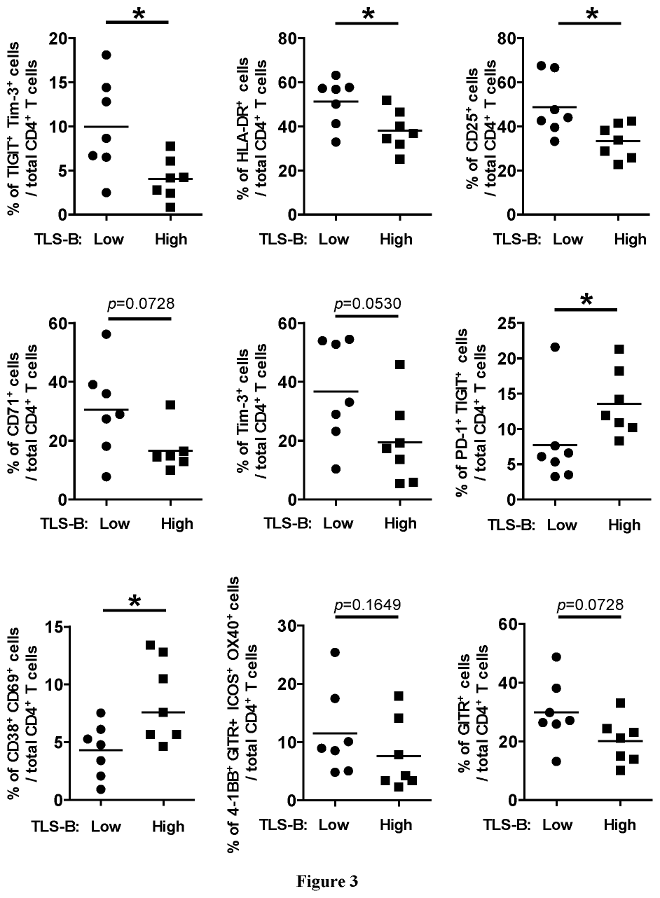

We next examined the relation between the presence of TLS-B cells (assessed by immunohistochemistry) and the phenotype of fresh intratumor CD4+ T cells (analyzed by flow cytometry) on the corresponding tumor biopsy. TLS-B cell density correlated positively with the percentages of activated [CD38+CD69+] CD4+ T cells and [PD-1+TIGIT+] CD4+ T cells (FIG. 2 and FIG. 3), and negatively correlated with the percentages of CD4+ T cells that were HLA-DR+, CD25+, CD71+, Tim-3+CD4+ T cells, and exhausted [TIGIT+Tim-3+], and, to a lesser extent, with the percentages of GITR+ and [4-1BB+GITR+ICOS+OX40+] CD4+ T cells (FIG. 2 and FIG. 3).

Remarkably, TLS-B cell density clustered with naive, CM, and EMRA CD4+ T cells (data not shown); this finding suggests that an active T cell homing, differentiation, and activation program takes place in TLS-B^{high} tumors. Conversely, most ICP and Treg markers, including CD25, GITR, CTLA-4, Tim-3 and TIGIT, were associated with clusters distinct and distant from the TLS-B cell cluster (data not shown).

These results, taken together, emphasize that high TLS-B cell density in the lung tumor-infiltrating CD4+ T cell compartment correlates positively with naive, CM, and activated cells and negatively with immunosuppressed T cells and Tregs.

A High Density of TLS-B Cells is Associated with Lower Treg Frequency in the NSCLC Tumor-Infiltrating CD4+ T Cell Compartment

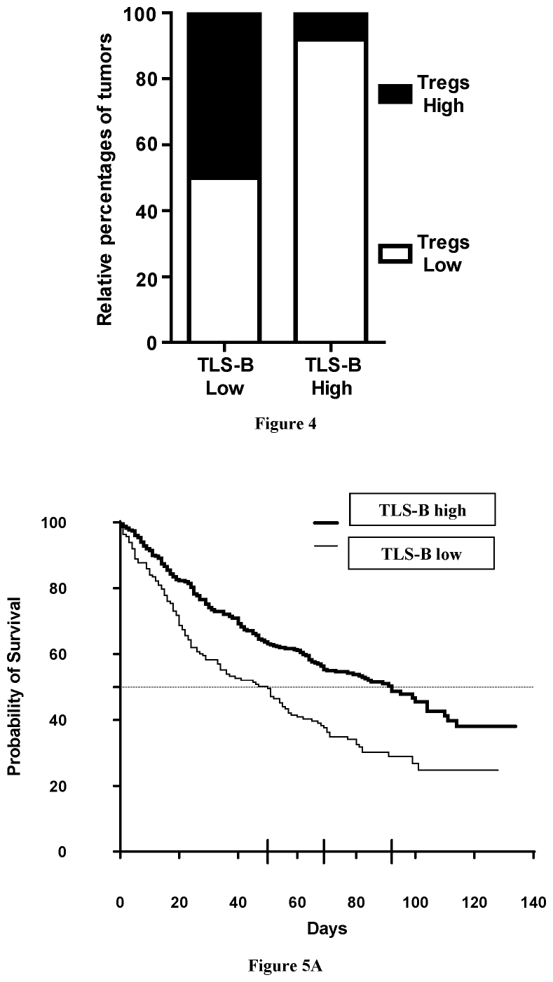

Because CD4+ Tregs strongly express CD25 (together with GITR and Tim-3),(18) we further characterized the phenotype of CD25+ TIL T cells relative to TLS-B cell density. We detected a population of CD25^{bright} CD4+ T cells co-expressing FoxP3 (data not shown) that was more frequent among total TIL CD4+ T cells in TLS-B^{low} versus TLS-B^{high} tumors (FIG. 4). A significant negative correlation was observed between the geomean of CD25 expressed by CD4+ T cells and TLS-B cell density in the corresponding tumor sample (data not shown), and to a lesser extent, between the percentage of CD25^{bright} CD4+ T cells and TLS-B cell density (data not shown). The percentage of Tregs among total CD4+ TIL T cells was higher in TLS-B^{low} tumors than TL S-B^{high} tumors (data not shown). This observation was confirmed on FFPE tumor sections, where 50% of TLS-B^{low} tumors were CD4+ Treg^{high}, composed with 8% in TLS-B high tumors (FIG. 4).

These results demonstrate that Tregs are most frequent in the CD4+ T cell compartment of tumors with a low density of TLS-B cells.

The Combination of TLS-B Cell and Treg Densities is a Strong Prognostic Indicator of Clinical Outcome in NSCLC Patients

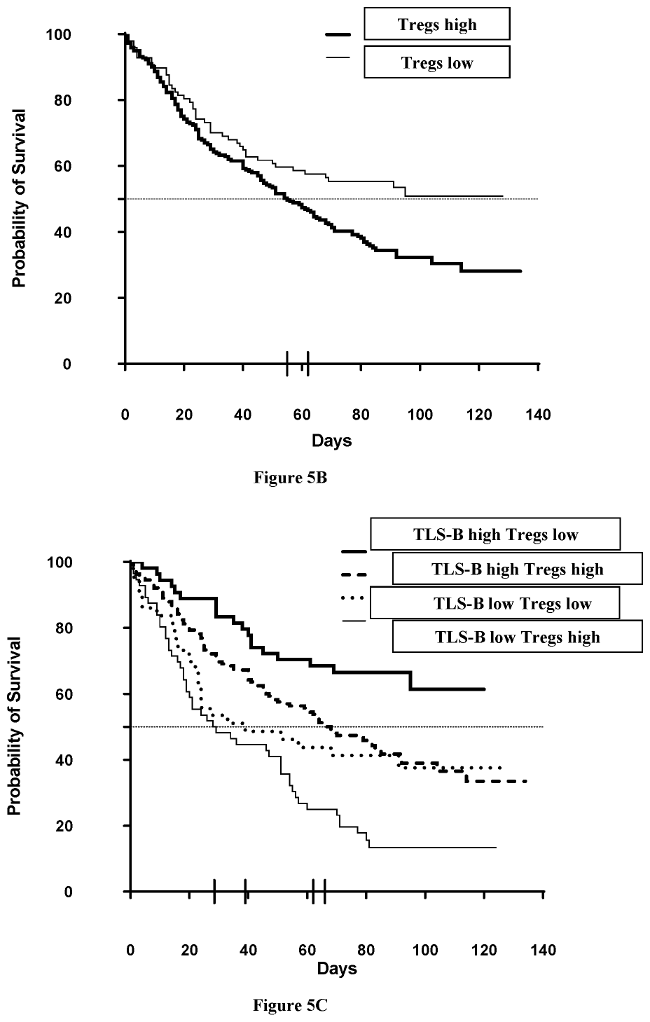

The prognostic value of CD20+ TLS-B cells was next investigated in a retrospective cohort (n=538 NSCLC, data not shown), through two distinct methods. We first confirmed that high TLS-B cell density was associated with prolonged survival [FIG. 5A, by applying the optimal cutoff with the median (2,12)]. The median values for TLS-B cell density did not differ significantly by tumor stage (data not shown) or histological subtype (data not shown). However, its favorable impact on survival was observed as early as stage I (A between best and worst median survival=24 months, p=0.005), peaked at stage II (A between best and worst median survivals=76 months, p=0.0007) but no longer existed at stages III-IV (A between best and worst median survival=7 months, p=0.1649) (data not shown). The prognostic value of TLS-B cell density was consistent for the ADC and SCC subtypes (A between the best and worst median survival=around 50 months, data not shown).

Next, we evaluated the prognostic value of CD3+FoxP3+ Tregs (n=330 patients, using the optimal cutoff data not shown). High Treg density was associated with a poor clinical outcome (Δ between best and worst median survival >78 months, FIG. 5B). Moreover, the combination of TLS-B cells and Tregs improved OS prediction over each individual parameter, identifying a group of NSCLC patients with the best outcome (TLS-B^{high}/Treg^{low}, median survival still unreached at 120 months, FIG. 5C). High TLS-B cell density was even able to counterbalance the deleterious impact of high Treg density on patient survival (median OS=66 months for TL S-B^{high}/Treg^{high} patients versus 28.5 months for TLS-B^{low}/Treg^{high} patients).

In univariate Cox regression analysis, patients' age (HR=2.239), pTNM stage (HR=2.841), and high Treg density (HR=1.617) were each significantly associated with poor OS, and high TLS-B cell density (HR=0.606) with long-term OS (Table 2). Multivariate Cox analysis showed that patients' age, pTNM stage, TLS-B cell density, and Tregs were independent prognostic factors (Table 3).

Taken together, these data show that high TLS-B cell density significantly and positively affects survival of NSCLC patients, and its evaluation makes it possible to better stratify patient prognosis, especially when combined with low Treg cell density.

### REFERENCES

Throughout this application, various references describe the state of the art to which this invention pertains. The disclosures of these references are hereby incorporated by reference into the present disclosure.
1. Galon J, Costes A, Sanchez-Cabo F, Kirilovsky A, Mlecnik B, Lagorce-Pages C, et al. Type, Density, and Location of Immune Cells Within Human Colorectal Tumors Predict Clinical Outcome. Science. 2006; 313:1960-4.
2. Germain C, Gnjatic S, Tamzalit F, Knockaert S, Remark R, Goc J, et al. Presence of B cells in tertiary lymphoid structures is associated with a protective immunity in patients with lung cancer. Am J Respir Crit Care Med. 2014; 189:832-44.
3. Fridman W H, Pages F, Sautes-Fridman C, Galon J. The immune contexture in human tumours: impact on clinical outcome. Nat Rev Cancer. 2012; 12:298-306.
4. Sautes-Fridman C, Lawand M, Giraldo N A, Kaplon H, Germain C, Fridman W H, et al. Tertiary Lymphoid Structures in Cancers: Prognostic Value, Regulation, and Manipulation for Therapeutic Intervention. Front Immunol. 2016; 7:407.
5. Dieu-Nosjean M-C, Antoine M, Danel C, Heudes D, Wislez M, Poulot V, et al. Long-Term Survival for Patients With Non-Small-Cell Lung Cancer With Intratumoral Lymphoid Structures. J Clin Oncol. 2008; 26:4410-7.
6. Germain C, Gnjatic S, Dieu-Nosjean M-C. Tertiary Lymphoid Structure-Associated B Cells are Key Players in Anti-Tumor Immunity. Front Immunol. 2015; 6:67.
7. Neyt K, Perros F, GeurtsvanKessel C H, Hammad H, Lambrecht B N. Tertiary lymphoid organs in infection and autoimmunity. Trends Immunol. 2012; 33:297-305.
8. Moyron-Quiroz J E, Rangel-Moreno J, Kusser K, Hartson L, Sprague F, Goodrich S, et al. Role of inducible bronchus associated lymphoid tissue (iBALT) in respiratory immunity. Nat Med. 2004; 10:927-34.
9. Cabrita R, Lauss M, Sanna A, Donia M, Skaarup Larsen M, Mitra S, et al. Tertiary lymphoid structures improve immunotherapy and survival in melanoma. Nature. 2020; 577:561-5.
10. Petitprez F, de Reynies A, Keung E Z, Chen T W-W, Sun C-M, Calderaro J, et al. B cells are associated with survival and immunotherapy response in sarcoma. Nature. 2020; 577:556-60.
11. Helmink B A, Reddy S M, Gao J, Zhang S, Basar R, Thakur R, et al. B cells and tertiary lymphoid structures promote immunotherapy response. Nature. 2020; 577:549-55.
12. Goc J, Germain C, Vo-Bourgais T K D, Lupo A, Klein C, Knockaert S, et al. Dendritic cells in tumor-associated tertiary lymphoid structures signal a Th1 cytotoxic immune contexture and license the positive prognostic value of infiltrating CD8+ T cells. Cancer Res. 2014; 74:705-15.
13. Zhu W, Germain C, Liu Z, Sebastian Y, Devi P, Knockaert S, et al. A high density of tertiary lymphoid structure B cells in lung tumors is associated with increased CD4(+) T cell receptor repertoire clonality. Oncoimmunology. 2015; 4:e1051922.
14. Braud V M, Biton J, Becht E, Knockaert S, Mansuet-Lupo A, Cosson E, et al. Expression of LLT1 and its receptor CD161 in lung cancer is associated with better clinical outcome. Oncoimmunology. 2018; 7:e1423184.
15. Altman D G, Lausen B, Sauerbrei W, Schumacher M. Dangers of using “optimal” cutpoints in the evaluation of prognostic factors. J Natl Cancer Inst. 1994; 86:829-35.
16. Brunner C, Sindrilaru A, Girkontaite I, Fischer K-D, Sunderkotter C, Wirth T. BOB.1/OBF.1 controls the balance of TH1 and TH2 immune responses. EMBO J. 2007; 26:3191-202.
17. Henderson J G, Opejin A, Jones A, Gross C, Hawiger D. CD5 instructs extrathymic regulatory T cell development in response to self and tolerizing antigens. Immunity. 2015; 42:471-83.
18. Sakaguchi S, Sakaguchi N, Asano M, Itoh M, Toda M. Immunologic self-tolerance maintained by activated T cells expressing IL-2 receptor alpha-chains (CD25). Breakdown of a single mechanism of self-tolerance causes various autoimmune diseases. J Immunol Baltim Md 1950. 1995; 155:1151-64.
19. Sakuishi K, Apetoh L, Sullivan J M, Blazar B R, Kuchroo V K, Anderson A C. Targeting Tim-3 and PD-1 pathways to reverse T cell exhaustion and restore anti-tumor immunity. J Exp Med. 2010; 207:2187-94.
20. Shakya A, Goren A, Shalek A, German C N, Snook J, Kuchroo V K, et al. Oct1 and OCA-B are selectively required for CD4 memory T cell function. J Exp Med. 2015; 212:2115-31.
21. Watanabe M, Takagi Y, Kotani M, Hara Y, Inamine A, Hayashi K, et al. Down-regulation of ICOS ligand by interaction with ICOS functions as a regulatory mechanism for immune responses. J Immunol Baltim Md 1950. 2008; 180:5222-34.
22. Chen L, Flies D B. Molecular mechanisms of T cell co-stimulation and co-inhibition. Nat Rev Immunol. 2013; 13:227-42.
23. Dawicki W, Bertram E M, Sharpe A H, Watts T H. 4-1BB and OX40 act independently to facilitate robust CD8 and CD4 recall responses. J Immunol Baltim Md 1950. 2004; 173:5944-51.
24. Ribas A, Wolchok J D. Cancer immunotherapy using checkpoint blockade. Science. 2018; 359:1350-5.
25. Marin-Acevedo J A, Dholaria B, Soyano A E, Knutson K L, Chumsri S, Lou Y. Next generation of immune checkpoint therapy in cancer: new developments and challenges. J Hematol Oncol J Hematol Oncol. 2018; 11:39.
26. McHugh R S, Whitters M J, Piccirillo C A, Young D A, Shevach E M, Collins M, et al. CD4(+)CD25(+) immunoregulatory T cells: gene expression analysis reveals a functional role for the glucocorticoid-induced TNF receptor. Immunity. 2002; 16:311-23.
27. Yan J, Zhang Y, Zhang J-P, Liang J, Li L, Zheng L. Tim-3 expression defines regulatory T cells in human tumors. PloS One. 2013; 8:e58006.
28. Knee D A, Hewes B, Brogdon J L. Rationale for anti-GITR cancer immunotherapy. Eur J Cancer Oxf Engl 1990. 2016; 67:1-10.
29. Hu D, Mohanta S K, Yin C, Peng L, Ma Z, Srikakulapu P, et al. Artery Tertiary Lymphoid Organs Control Aorta Immunity and Protect against Atherosclerosis via Vascular Smooth Muscle Cell Lymphotoxin β Receptors. Immunity. 2015; 42:1100-15.
30. Li W, Bribriesco A C, Nava R G, Brescia A A, Ibricevic A, Spahn J H, et al. Lung transplant acceptance is facilitated by early events in the graft and is associated with lymphoid neogenesis. Mucosal Immunol. 2012; 5:544-54.
31. Fletcher A L, Lukacs-Kornek V, Reynoso E D, Pinner S E, Bellemare-Pelletier A, Curry M S, et al. Lymph node fibroblastic reticular cells directly present peripheral tissue antigen under steady-state and inflammatory conditions. J Exp Med. 2010; 207:689-97.
32. Cohen I N, Guidi C J, Tewalt E F, Qiao H, Rouhani S J, Ruddell A, et al. Lymph node-resident lymphatic endothelial cells mediate peripheral tolerance via Aire-independent direct antigen presentation. J Exp Med. 2010; 207:681-8.
33. Warren K J, Iwami D, Harris D G, Bromberg J S, Burrell B E. Laminins affect T cell trafficking and allograft fate. J Clin Invest. 2014; 124:2204-18.
34. Baptista A P, Roozendaal R, Reijmers R M, Koning J J, Unger W W, Greuter M, et al. Lymph node stromal cells constrain immunity via MHC class II self-antigen presentation. eLife. 2014; 3.
35. Costa A, Kieffer Y, Scholer-Dahirel A, Pelon F, Bourachot B, Cardon M, et al. Fibroblast Heterogeneity and Immunosuppressive Environment in Human Breast Cancer. Cancer Cell. 2018; 33:463-479.e10.
36. Hindley J P, Jones E, Smart K, Bridgeman H, Lauder S N, Ondondo B, et al. T-cell trafficking facilitated by high endothelial venules is required for tumor control after regulatory T-cell depletion. Cancer Res. 2012; 72:5473-82.
37. Joshi N S, Akama-Garren E H, Lu Y, Lee D-Y, Chang G P, Li A, et al. Regulatory T Cells in Tumor-Associated Tertiary Lymphoid Structures Suppress Anti-tumor T Cell Responses. Immunity. 2015; 43:579-90.
38. Tsuji M, Suzuki K, Kitamura H, Maruya M, Kinoshita K, Ivanov I I, et al. Requirement for lymphoid tissue-inducer cells in isolated follicle formation and T cell-independent immunoglobulin A generation in the gut. Immunity. 2008; 29:261-71.
39. Gu-Trantien C, Loi S, Garaud S, Equeter C, Libin M, de Wind A, et al. CD4+ follicular helper T cell infiltration predicts breast cancer survival. J Clin Invest. 2013; 123:2873-92.
40. Couillault C, Germain C, Dubois B, Kaplon H. Identification of Tertiary Lymphoid Structure-Associated Follicular Helper T Cells in Human Tumors and Tissues. Methods Mol Biol Clifton N J. 2018; 1845:205-22.
41. Croft M. Co-stimulatory members of the TNFR family: keys to effective T-cell immunity? Nat Rev Immunol. 2003; 3:609-20.
42. Weinberg A D, Rivera M M, Prell R, Morris A, Ramstad T, Vetto J T, et al. Engagement of the OX-40 receptor in vivo enhances antitumor immunity. J Immunol Baltim Md 1950. 2000; 164:2160-9.
43. Melero I, Shuford W W, Newby S A, Aruffo A, Ledbetter J A, Hellström K E, et al. Monoclonal antibodies against the 4-1B B T-cell activation molecule eradicate established tumors. Nat Med. 1997; 3:682-5.
44. Cabo M, Offringa R, Zitvogel L, Kroemer G, Muntasell A, Galluzzi L. Trial Watch: Immunostimulatory monoclonal antibodies for oncological indications. Oncoimmunology. 2017; 6:e1371896.
45. Sharma P, Allison J P. Immune checkpoint targeting in cancer therapy: toward combination strategies with curative potential. Cell. 2015; 161:205-14.
46. Detterbeck F C, Boffa D J, Tanoue L T. The new lung cancer staging system. CHEST J. 2009; 136:260-71.
47. Brambilla E, Travis W D, Colby T V, Corrin B, Shimosato Y. The new World Health Organization classification of lung tumours. Eur Respir J. 2001; 18:1059-68.

1. A method for determining the TLS (Tertiary Lymphoid Structures) status and/or predicting the survival time of a patient suffering from a lung cancer and treating the patient comprising i) determining in a sample obtained from said patient an expression level of at least one marker selected from the group consisting of CD69+, CM, CD8+, CD40L+, CD86+, TIM-3+, CD4+ and PD-1 and ii) administering an anti-cancer agent to a subject identified as having an expression level lower than a corresponding predetermined reference value.

2. (canceled)

3. The method of claim 1, wherein the expression level of the markers or of a combination of the markers CD69 or CM and CD8+ or CD40L+ and CD8+ or CD86+ is determined.

4. The method according to claim 1 wherein the expression level of a combination of markers TIM-3+ and CD4+ or TIM-3+ and CD8+ or TIM-3+, PD-1+ and CD8+ is determined.

5. The method according to claim 1, wherein the sample is peripheral-blood, serum, plasma or saliva.

6. A method for determining the TLS (Tertiary Lymphoid Structures) status and/or predicting the survival time of a patient suffering from a lung cancer and treating the patient comprising i) determining in a sample obtained from a tumor of the patient an expression level of at least one marker selected from the group consisting of CD4+, TIGIT+, TIM-3+, HLA-DR+, CD25+, CD71+, PD-1+, 4-1BB+, GITR+, ICOS+, OX40+, CD38+ and CD69+ and ii) administering an anti-cancer agent to a subject identified as having an expression level that is lower than a corresponding predetermined reference value.

7. (canceled)

8. (canceled)

9. The method of claim 6, wherein the expression level of a combination of markers CD4+, PD-1+ and TIGIT+, or CD4+, CD38+ and CD69+ is determined.

10. The method of claim 6, wherein the expression level of a combination of markers CD4+, TIGIT+ and TIM3+, or CD4+ and HLA-DR+, or CD4+ and CD25+, or CD4+ and CD71+, or CD4+ and TIM3+, or CD4+, 4-1BB+, GITR+, ICOS+ and OX40+, or CD4+ and GITR+, or CD4+, PD1+, TIGIT+ and TIM-3+ is determined.

11. (canceled)

12. (canceled)

13. (canceled)
